(19) United States
(12) Patent Application Publication
(10) Pub. No.: US 2020/0239959 A1
(43) Pub. Date: Jul. 30, 2020

(54) Plasma MicroRNA Markers of Upper Limb Recovery Following Human Stroke

(71) Applicant: Georgetown University, Washington, DC (US)

(72) Inventors: Matthew A. Edwardson, McLean, VA (US); Xiaogang Zhong, College Park, MD (US); Massimo S. Fiandaca, Millersville, MD (US); Howard J. Federoff, Irvine, CA (US); Amrita K. Cheema, Potomac, MD (US); Alexander W. Dromerick, Washington, DC (US)

(73) Assignee: Georgetown University, Washington, DC (US)

(21) Appl. No.: 16/751,700

(22) Filed: Jan. 24, 2020

Related U.S. Application Data
(60) Provisional application No. 62/796,568, filed on Jan. 24, 2019.

(51) Int. Cl. C12Q 1/6883

(52) U.S. Cl. CPC C12Q 1/6883; C12Q 2600/178; G01N 2800/54; G01N 2800/50

(57) ABSTRACT

Methods of determining if a subject has an increased risk of poor recovery after suffering a stroke and methods of treating a subject recovering from a stroke. The methods comprise analyzing at least one plasma sample taken from the subject to assess a microRNA (miRNA) profile of the subject and comparing the subject's miRNA profile with a normal miRNA profile, to determine if the subject's miRNA profile is altered compared to a normal miRNA profile. An alteration of the subject's miRNA profile is indicative that the subject has an increased risk of poor recovery after suffering a stroke.

### CROSS-REFERENCE TO RELATED APPLICATIONS

This application claims the benefit of U.S. Provisional Application No. 62/796,568 filed on Jan. 24, 2019, the entirety of which is herein incorporated by reference.

### STATEMENT REGARDING FEDERALLY SPONSORED RESEARCH OR DEVELOPMENT

Part of the work performed during development of this invention utilized U.S. Government funds under National Institutes of Health, Grant Nos. P30-CA051008, U54TR001366-01, and UL1TR000101. The U.S. Government has certain rights in this invention.

### FIELD OF THE INVENTION

The present invention relates to methods of determining if a subject has an increased risk of poor recovery after suffering a stroke. The methods comprise analyzing at least one plasma sample taken from the subject to assess a microRNA (miRNA) profile of the subject and comparing the subject's miRNA profile with a normal miRNA profile, to determine if the subject's miRNA profile is altered compared to a normal miRNA profile. An alteration of the subject's miRNA profile is indicative that the subject has an increased risk of poor recovery after suffering a stroke.

### BACKGROUND OF THE INVENTION

Ribonucleic acid (RNA) species include microRNAs (miRNAs or miRs), which are small non-coding ˜21 residue RNA species. Initially transcribed from nuclear DNA as a primary miRNA (pri-miRNA) transcript, pri-miRNA is then processed within the nucleus to form precursor miRNA (pre-miRNAs) that are transported to the cytoplasm, where they are further processed to form the unique miRNA species that interact and influence messenger RNA (mRNA) expression. The human genome encodes over 2000 miRNAs, which help regulate the expressed transcripts of roughly half of all genes. MiRNAs function by either degrading mRNA directly (along with a cleavage protein) or through binding to RNA-induced silencing complexes (RISCs) that inhibit/prevent mRNA translation and thereby decrease the synthesis of specific proteins. MiRNAs are quite stable in plasma, where they are protected from enzymatic degradation by transport within extracellular vesicles (EVs) and high density lipoproteins. As intraluminal EV cargos, short nucleotide sequences, like miRNAs, are capable of being transported across the blood-brain barrier. Dysregulated plasma miRNAs have also been identified in various forms of cancer and neurological diseases such as Alzheimer's, multiple sclerosis, and stroke.

While many investigators have studied miRNA expression related to the acute phase of stroke (during the first 72 hours) in both animal models and humans, few have investigated miRNAs during the recovery phase. Vijayan and colleagues recently discovered four stroke-related miRNAs (PC-3p-57664, PC-5p-12969, miR-122-5p, and miR-211-5p) that are dysregulated not only in human acute stroke serum samples, but also in human post-mortem ischemic brain tissue and acute mouse stroke models. Within 24-48 hours of a middle cerebral artery occlusion (MCAO) in rodents, there is upregulation of brain-specific miR-124a in brain parenchyma and peripheral blood. Interestingly, a separate study found that miR-124a was downregulated seven days post-MCAO in the subventricular zone (SVZ), which was thought to promote neural progenitor cell differentiation during neural repair. Other preclinical investigators found that miR-146a is upregulated between 0-7 days post-MCAO and may contribute to oligodendrocyte precursor cell differentiation in the SVZ19. To our knowledge, there are no prior studies of miRNA expression during the window of maximum spontaneous biological recovery from stroke in humans (˜72 hours to three months post-stroke). This sensitive period of heightened neural plasticity is characterized by waves of differential gene expression that are associated with axonal sprouting over the first month, and an increase in synaptic density. The differential gene expression during the sensitive period is regulated, at least in part, by miRNAs. Thus, understanding the expression pattern of miRNA following a stroke can help identify those patients that may be susceptible to a poor recovery, as well as optimize the treatments for patients recovering from stroke.

### SUMMARY OF THE INVENTION

The present invention relates to methods of determining if a subject has an increased risk of poor recovery after suffering a stroke. The methods comprise analyzing at least one plasma sample taken from the subject to assess an miRNA profile of the subject and comparing the subject's miRNA profile with a normal miRNA profile, to determine if the subject's miRNA profile is altered compared to a normal miRNA profile. An alteration of the subject's miRNA profile is indicative that the subject has an increased risk of poor recovery after suffering a stroke.

The present invention also relates to methods of treating a subject who is recovering from a stroke, or a subject who has an increased risk of a poor recovery from a stroke. The methods may comprise (a) determining whether the subject has an increased risk of poor recovery from a stroke by analyzing at least one plasma sample taken from the subject to assess an miRNA profile of the subject and comparing the subject's miRNA profile with a normal miRNA profile, wherein a difference in the subject's miRNA profile compared to a normal miRNA profile is indicative that the subject has an increased risk of poor recovery from the stroke; and (b) administering a treatment for recovery from the stoke when the subject is determined to have an increased risk of poor recovery from the stroke.

The present invention further relates to methods of treating a subject who is recovering from a stroke, in which the methods may comprise (a) determining whether the subject has an increased risk of poor recovery from a stroke by analyzing at least one plasma sample taken from the subject to assess an miRNA profile of the subject and comparing the subject's miRNA profile with a normal miRNA profile, wherein a difference in the subject's miRNA profile compared to a normal miRNA profile is indicative that the subject has an increased risk of poor recovery from the stroke; and (b) administering (i) a treatment effective for poor recovery from the stoke when the subject is determined to have an increased risk of poor recovery from the stroke; or (ii) a treatment effective for normal or good recovery from the stoke when the subject is determined to not have an increased risk of poor recovery from the stroke.

### BRIEF DESCRIPTION OF THE DRAWINGS

FIG. 1 depicts the fold-change for miRNAs with significant differential expression between participants with good recovery of the upper limb, as determined by at least a six-point increase in the Action Research Arm Test (ARAT) score from baseline to six months (ΔARAT≥6), versus poor recovery of the upper limb (ΔARAT<6). Error bars represent standard deviation.

FIG. 2 depicts (A) receiver operating characteristic (ROC) curve for good (ΔARAT≥6) versus poor (ΔARAT<6) recovery using a combination of five miRNAs: miR-581, miR-519b-3p, miR-941, miR-449b, and miR-616; and (B) predicted class probabilities for the five miRNA predictive panel, demonstrating 25 correctly classified and two misclassified participants. The two misclassified participants are labeled by their respective AARAT scores.

### DETAILED DESCRIPTION OF THE INVENTION

Although blood-based biomarkers for neurological health and disease are gaining recognition, there are currently no clinically relevant blood-based biomarkers for neural repair in humans. Such biomarkers would be extremely valuable in identifying the sensitive period of heightened plasticity known to occur after a stroke, and to allow optimal timing of rehabilitation strategies. Human blood-based biomarkers may also provide insights into specific brain repair biology and help drive translational discoveries using preclinical animal models. The exploratory clinical study described in the Examples below was the first step in determining whether plasma miRNAs might hold promise as stroke recovery biomarkers, recognizing the limitations of such a reductionistic approach and likely enhancement through future use of multi-omic assessments. Through a comparison of plasma from stroke recovery participants with good versus poor recovery, however, miRNAs were identified that showed significant differential expression between the groups. None of these miRNAs had been previously reported in human stroke or rodent stroke models.

Some of the miRNAs identified are notably dysregulated in various forms of cancer, including two (miR-520g, miR-524) that affect proliferation of gliomas. The association with cancer may not be coincidental, as the molecular machinery for tumor proliferation and regenerative axonal sprouting often overlap. None of these miRNAs overlapped with the acute stroke-related miRNAs recently found to be shared between humans and rodents. The pathway analysis also suggests that the miRNAs converge on cancer-related and neural repair pathways. Pathways like axonal guidance and glioma formation point directly to neural parenchymal involvement, whereas others, such as WNT signaling and pluripotency of stem cells, are less specific to the central nervous system, but could contribute to neural repair.

The present invention relates to methods of determining if a subject has an increased risk of poor recovery after suffering a stroke. The methods comprise analyzing at least one plasma sample taken from the subject to assess an miRNA profile of the subject and comparing the subject's miRNA profile with a normal miRNA profile, to determine if the subject's miRNA profile is altered compared to a normal miRNA profile. An alteration of the subject's miRNA profile is indicative that the subject has an increased risk of poor recovery after suffering a stroke.

The present invention also relates to methods of treatment. In some embodiments, the methods are for treating a subject who is recovering from a stroke. In some embodiments, the methods are for treating a subject who has an increased risk of a poor recovery from a stroke. The methods may comprise (a) determining whether the subject has an increased risk of poor recovery from a stroke by analyzing at least one plasma sample taken from the subject to assess an miRNA profile of the subject and comparing the subject's miRNA profile with a normal miRNA profile, wherein a difference in the subject's miRNA profile compared to a normal miRNA profile is indicative that the subject has an increased risk of poor recovery from the stroke; and (b) administering a treatment for recovery from the stoke when the subject is determined to have an increased risk of poor recovery from the stroke. In some embodiments, the methods may comprise (a) determining whether the subject has an increased risk of poor recovery from a stroke by analyzing at least one plasma sample taken from the subject to assess an miRNA profile of the subject and comparing the subject's miRNA profile with a normal miRNA profile, wherein a difference in the subject's miRNA profile compared to a normal miRNA profile is indicative that the subject has an increased risk of poor recovery from the stroke; and (b) administering (i) a treatment effective for poor recovery from the stoke when the subject is determined to have an increased risk of poor recovery from the stroke; or (ii) a treatment effective for normal or good recovery from the stoke when the subject is determined to not have an increased risk of poor recovery from the stroke.

The treatments administered to the subject may comprise rehabilitation therapies effective to help in the recovery from a stroke.

As used herein, the term subject or “test subject” indicates a mammal, in particular a human or non-human primate.

In some embodiments, a “poor recovery” or “good recovery” may be based on how quickly, to what extent, or both, a subject can be restored of one or more functions that have diminished or were lost because of the stroke, or can be relieved of one or more symptoms or ailments as a result of the stroke, as compared to a normal recovery. In certain embodiments, a “poor recovery” or “good recovery” following a stroke may be characterized by different techniques known in the art. For example, “poor recovery” or “good recovery” may be based on the results using one or more of Action Research Arm Test (ARAT), one or more elements of the NIH Stroke Scale (NIHSS), modified Rankin Scale, Barthel Index, Functional Independence Measure, Folstein Mini-Mental State Examination, Fugl-Meyer, Motor Assessment Scale, Berg Balance Assessment, Boston Diagnostic Aphasia Examination, etc.

“Normal recovery” may mean a recovery time and/or extent of recovery that would be expected in the art based on the nature of the stroke, the age and condition of the subject, etc. In some embodiments, a “normal” recovery may mean an average recovery time and/or average extent of recovery. “Average” may be a range. In such embodiments, “poor recovery” would be a recovery that is below normal, e.g., longer than the normal recovery time, does not reach the normal extent of recovery, or a combination thereof. In some embodiments, “good recovery” may be a recovery that is above normal, e.g., shorter than the normal recovery time, exceeds the normal extent of recovery, or a combination thereof. In some embodiments, “good recovery” may mean the same as, or may encompass a normal recovery, e.g., a recovery time that is the same as and/or shorter than a normal recovery time, an extent of recovery that is the same as and/or exceeds a normal extent of recovery, or a combination thereof.

An “miRNA profile” may mean a combination of miRNAs of a subject found in the peripheral blood or portions thereof, such as but not limited to plasma or serum. The miRNA profile may be a collection of measurements, such as but not limited to a quantity or concentration or expression level, for individual miRNAs taken from a test sample of the subject. Thus, in some embodiments, the assessment of an miRNA profile comprises assessing expression levels of one or more miRNAs.

Examples of test samples or sources of components for the miRNA profile include, but are not limited to, biological fluids, which can be tested by the methods of the present invention described herein, and include but are not limited to whole blood, such as but not limited to peripheral blood, serum, plasma, cerebrospinal fluid, urine, amniotic fluid, lymph fluids, and various external secretions of the respiratory, intestinal and genitourinary tracts, tears, saliva, milk, white blood cells, myelomas, and the like. Test samples to be assayed also include but are not limited to tissue specimens including normal and abnormal tissue. Techniques to assay levels of individual components of the miRNA profile from test samples are well known to the skilled technician, and the invention is not limited by the means by which the components are assessed.

The assessment of the levels of the individual components of the miRNA profile can be expressed as absolute or relative values and may or may not be expressed in relation to another component, a standard or internal standard, or a different RNA molecule known to be in the sample. If the levels are assessed as relative to a standard or internal standard, the standard may be added to the test sample prior to, during, or after sample processing.

To assess levels of the individual components of the miRNA profile, a sample is taken from the subject. The sample may or may not be processed prior to assaying levels of the components of the miRNA profile. For example, whole blood may be taken from an individual and the blood sample may be processed, e.g., centrifuged, to isolate plasma or serum from the blood. The sample may or may not be stored, e.g., frozen, prior to processing or analysis.

In embodiments of the invention, the miRNA profile may comprise one or more miRNAs, or the measurement or quantification (such as expression levels) of one or more miRNAs, selected from miR-371-3p, miR-524, miR-520g, miR-1255a, miR-453, miR-941, miR-449b, miR-581, miR-583, miR-519b, and miR-616. In certain embodiments, the miRNA profile comprises miR-581, miR-519b-3p, miR-941, miR-449b, and miR-616. In certain embodiments, the miRNA profile comprises the measurement or quantification (such as expression levels) of miR-581, miR-519b-3p, miR-941, miR-449b, and miR-616.

In some embodiments, the comparison of an miRNA profile of the subject with a normal miRNA profile may comprise comparing the expression level of each of the miRNAs of the subject's miRNA profile with the expression level of each of the miRNAs of the normal miRNA profile. In some embodiments, the comparison of an miRNA profile of the subject with a normal miRNA profile may comprise determining whether the expression level of each of the miRNAs of the subject's miRNA profile is higher or lower than the expression level of each of the miRNAs of the normal miRNA profile.

In some embodiments, the determination of whether the expression level of an miRNA of the subject's miRNA profile is higher or lower than the expression level of the miRNA of the normal miRNA profile may be performed by assessing the absolute difference in the miRNA expression level of the subject as compared to the normal miRNA expression level. In some embodiments, the determination of whether the expression level of an miRNA of the subject's miRNA profile is higher or lower than the expression level of the miRNA of the normal miRNA profile may be performed by assessing the relative difference (e.g., percent difference) in the subject's miRNA expression level as compared to normal miRNA expression level.

In some embodiments, a difference in the subject's miRNA profile as compared to a normal miRNA profile may be indicative that the subject has an increased risk of poor recovery from the stroke when the expression level of at least one of miR-3′71-3p, miR-524, miR-520g, miR-1255a, miR-453, and miR-583 of the subject's miRNA profile is lower as compared to the expression level of the normal miRNA profile. In some embodiments, a difference in the subject's miRNA profile as compared to a normal miRNA profile may be indicative that the subject has an increased risk of poor recovery from the stroke when the expression level of at least two of miR-371-3p, miR-524, miR-520g, miR-1255a, miR-453, and miR-583 of the subject's miRNA profile is lower as compared to the expression level of the normal miRNA profile. In some embodiments, a difference in the subject's miRNA profile as compared to a normal miRNA profile may be indicative that the subject has an increased risk of poor recovery from the stroke when the expression level of at least three of miR-3′71-3p, miR-524, miR-520g, miR-1255a, miR-453, and miR-583 of the subject's miRNA profile is lower as compared to the expression level of the normal miRNA profile. In some embodiments, a difference in the subject's miRNA profile as compared to a normal miRNA profile may be indicative that the subject has an increased risk of poor recovery from the stroke when the expression level of at least four of miR-371-3p, miR-524, miR-520g, miR-1255a, miR-453, and miR-583 of the subject's miRNA profile is lower as compared to the expression level of the normal miRNA profile. In some embodiments, a difference in the subject's miRNA profile as compared to a normal miRNA profile may be indicative that the subject has an increased risk of poor recovery from the stroke when the expression level of at least five of miR-371-3p, miR-524, miR-520g, miR-1255a, miR-453, and miR-583 of the subject's miRNA profile is lower as compared to the expression level of the normal miRNA profile. In some embodiments, a difference in the subject's miRNA profile as compared to a normal miRNA profile may be indicative that the subject has an increased risk of poor recovery from the stroke when the expression level of each of miR-371-3p, miR-524, miR-520g, miR-1255a miR-453, and miR-583 of the subject's miRNA profile is lower as compared to the expression level of the normal miRNA profile.

In some embodiments, a difference in the subject's miRNA profile as compared to a normal miRNA profile may be indicative that the subject has an increased risk of poor recovery from the stroke when the expression level of at least one of miR-941, miR-449b, miR-581, miR-519b, or miR-616 of the subject's miRNA profile is higher as compared to the expression level of the normal miRNA profile. In some embodiments, a difference in the subject's miRNA profile as compared to a normal miRNA profile may be indicative that the subject has an increased risk of poor recovery from the stroke when the expression level of at least two of miR-941, miR-449b, miR-581, miR-519b, or miR-616 of the subject's miRNA profile is higher as compared to the expression level of the normal miRNA profile. In some embodiments, a difference in the subject's miRNA profile as compared to a normal miRNA profile may be indicative that the subject has an increased risk of poor recovery from the stroke when the expression level of at least three of miR-941, miR-449b, miR-581, miR-519b, or miR-616 of the subject's miRNA profile is higher as compared to the expression level of the normal miRNA profile. In some embodiments, a difference in the subject's miRNA profile as compared to a normal miRNA profile may be indicative that the subject has an increased risk of poor recovery from the stroke when the expression level of at least four of miR-941, miR-449b, miR-581, miR-519b, or miR-616 of the subject's miRNA profile is higher as compared to the expression level of the normal miRNA profile. In some embodiments, a difference in the subject's miRNA profile as compared to a normal miRNA profile may be indicative that the subject has an increased risk of poor recovery from the stroke when the expression level of each of miR-941, miR-449b, miR-581, miR-519b, or miR-616 of the subject's miRNA profile is higher as compared to the expression level of the normal miRNA profile.

In some embodiments, the comparison of the miRNA profile of the subject with the normal miRNA profile may be performed by, for each miRNA profile, assigning a single value, number, factor, or score given as an overall collective value to the individual miRNA components of the profile, or to categorical components, e.g., miRNAs associated with axon guidance, miRNAs associated with WNT signaling, etc.. For example, if each miRNA is assigned a value, such as above, the miRNA profile value may simply be the overall score of each individual or categorical value. For instance, if four of the components of the miRNA profile are involved axon guidance, and two of those components are assigned values of “−2” and two are assigned values of “+1,” the axon guidance portion of the miRNA profile in this example would be −2, with a normal value being, for example, “0.” In this manner, the miRNA profile value could be a useful single number or score, the actual value or magnitude of which could be an indication of the actual risk of poor recovery from a stroke, e.g., the “more negative” the value, the less the risk of experiencing poor stroke recovery.

In some embodiments, the comparison of the miRNA profile of the subject with the normal miRNA profile may be performed by, for each miRNA profile, assigning a series of values, numbers, factors, or scores given to the individual components of the overall profile. In other embodiments, each miRNA profile may be assigned a combination of values, numbers, factors or scores given to individual components of the profile as well as values, numbers, factors or scores collectively given to a group of components, such as a axon guidance portion, a WNT signaling portion, etc. In another example, each miRNA profile value may comprise or consist of individual values, number, factors, or scores for specific component as well as values, numbers, factors, or scores for a group on components.

In some embodiments, the comparison of the miRNA profile of the subject with the normal miRNA profile may be performed by, for each miRNA profile, assigning a “combined miRNA index” based on individual values from the miRNAs that are used to develop a single score, and which may utilize weighted scores from the individual component values reduced to a diagnostic number value. The combined miRNA index may also be generated using non-weighted scores from the individual component values. When the combined miRNA index exceeds (or drops below) a specific threshold level, determined by a range of values developed similarly from control or normal subjects or subjects who experienced good recovery from a stroke, the individual has a low risk, or lower than normal risk, of experiencing a poor recovery from a stroke, whereas maintaining a normal range value of the combined miRNA index may indicate a normal risk of experiencing poor recovery from a stroke. In this embodiment, the threshold value would be or could be set by the combined miRNA index from one or more normal subjects or subjects who experienced good recovery from a stroke.

In some embodiments, a “normal” miRNA profile or a “normal” expression level of an miRNA may be an miRNA profile or an miRNA expression level measured in the general population or a representative of the general population. In certain embodiments, a “normal” miRNA profile or a “normal” expression level of an miRNA may be an miRNA profile or an miRNA expression level of individuals who experienced or are experiencing a normal, good, favorable, and/or full recovery from a stroke.

Treatment for subjects who are recovering from a stroke, who have an increased risk of a poor recovery from a stroke, or who do not have an increased risk of a poor recovering from a stroke, may comprise applying rehabilitation therapies, including rehabilitation therapies effective for subjects who are poorly recovering from strokes. Such therapies may include, but are not limited to, physical therapy, occupational therapy, speech-language therapy, hearing therapy, recreational therapy, nutritional care, psychiatric/psychological therapy, or a combination thereof. Treatments may also include, but are not limited to, measures to reduce the risk of another stroke, such as adopting healthy lifestyle habits; controlling risk factors such as high blood pressure, smoking, and atrial fibrillation; and/or taking medication to lower high blood pressure, manage atrial fibrillation, and reduce the chances of forming a clot.

Treatments that are effective for poor recovery from a stroke may be known in the art, and can include rehabilitation therapies that are focused on restoring more basic functions, that are slower to progress to restoring more difficult tasks, that require greater involvement of medical or therapeutic assistance, etc. Treatments that are effective for normal or good recovery from a stroke may be known in the art as well, and can include rehabilitation therapies that are focused on restoring more advanced functions, that are more intense, that follows a more aggressive timeline, etc.

### EXAMPLES

### Example 1

A Critical Periods After Stroke Study (CPASS) was performed at the MedStar National Rehabilitation Hospital (Washington, DC). The study was approved by the MedStar Health Research Institute IRB (approval #2014-065) and carried out according to their guidelines and regulations; all participants provided written informed consent. Plasma samples were collected from 27 CPASS participants at the time of enrollment. Arm motor function was assessed at baseline and 6 months post-stroke using ARAT.

Inclusion criteria featured the following: ischemic or hemorrhagic stroke; age ≥21; NIHSS arm motor item ≥1; at least a minimal level of preserved function in the hemiparetic arm; Short Blessed Memory Orientation and Concentration Test score ≤8; follows two-step commands; no prior injury to limb limiting use; and pre-stroke modified Rankin Score <2. Exclusion criteria featured the following: unable to give informed consent; history of prior stroke with persistent hemiparesis or other disabling neurologic condition; hemispatial neglect (asymmetry >3 on Mesulam Symbol Cancellation Test); NIHSS sensory item score of 2; NIHSS limb ataxia item ≥1; active or prior psychosis or substance abuse; life expectancy <1 year; and received botulinum toxin injection within six months.

Fasting blood samples were collected by venipuncture at the baseline study assessment between 7-9 AM in EDTA-tubes (Cardinal Health, Ohio, USA). Collecting blood samples near the time of inpatient rehabilitation admission, as opposed to the acute hospitalization, attempted to avoid capturing molecular changes related to the initial injury and attempted to capture changes associated with spontaneous biological recovery. The blood samples were thoroughly mixed, placed on ice and centrifuged at 2600 RPM for ten minutes at 20° C. Plasma was carefully removed via pipette, being careful not to disturb the adjacent buffy coat. Plasma was collected in 750 μL aliquots and frozen at −80° C. until ready for analysis.

Total RNA, including miRNAs and other small RNA molecules, was isolated from 200 μl of plasma and extracted using the Qiagen miRNeasy Serum/Plasma Kit (QIAGEN, Valencia, Calif.), according to the manufacturer's instructions. After extraction, the RNA concentration and purity (OD260/280) were measured using the NanoDrop ND-1000 spectrophotometer (Thermo Fischer Scientific, Waltham, Mass.), and the RNA integrity number (RIN) was determined using an Agilent 2100 Bioanalyzer Instrument (Agilent, Santa Clara, Calif., USA). Reverse-transcription (RT) was carried out using input amounts of 33 nanograms (ng) of total RNA, with APPLIED BIOSYSTEMS MEGAPLEX™ RT Primers, Human Pool A and B v3.0, and enzyme kit. This was followed by a subsequent step of pre-amplification (12 cycles) using MEGAPLEX™ PreAmp Primers, Human Pool A and B v3.0, to enhance assay sensitivity as recommended by the manufacturer (Life Technologies, Carlsbad, Calif.). Prior to quantitative reverse transcription-polymerase chain reaction (qRT-PCR), complementary DNAs (cDNAs) were loaded onto 384-well format miRNA assays plates (Taqman Array Human MicroRNA A+B Cards, V3.0, Applied Biosystems, Foster City, Calif.). Subsequently, qRT-PCR was performed on a 7900HT Real-Time PCR System (Applied Biosystems, Foster City, Calif.).

Good recovery was defined as a change (Δ) in the ARAT score from baseline (median 19 days post-stroke) to six months ≥6. A change of six points was chosen because prior rehabilitation investigators have determined that this is the minimum level of change on the ARAT scale that is clinically meaningful to stroke patients. After data pre-processing, the miRNA expression values were normalized with log transformation, to stabilize the variance, followed by quantile normalization, to make the empirical distribution of intensities similar across samples. Differential expression between patient groups was assessed using independent samples or Wilcoxon-Mann-Whitney U tests. Significance (p) values are reported after adjustment for multiple comparisons, using the false discovery rate (FDR) approach by Benjamini and Hochberg. MiRNAs with differential expression between the two groups, using FDR-corrected p<0.05, were considered significant. Pearson correlations were determined using the ΔARAT for each individual participant and the expression of each significant miRNA. Analysis was performed using a custom algorithm developed in the ‘R’ programming language. Receiver operating characteristic curve analysis was performed using MetaboAnalyst v4.0, available on the world wide web at metaboanalyst.ca/faces/home.xhtml.

### Example 2

Twenty-two of 27 clinical participants showed good recovery, as determined by at least a six-point increase in the Action Research Arm Test (ARAT) score from baseline to 6 mo., while the remaining five participants displayed poor recovery (ΔARAT<6). Characteristics for the 27 participants in the good and poor recovery groups are described in Table 1. Despite the small number of participants with poor recovery, the two groups were fairly well matched with regard to gender, cardiovascular comorbidities, and time from stroke onset to baseline blood collection (median 19 days for all 27 participants). The poor recovery group was typically older than the good recovery group (median 72 vs. 62.5 respectively) and had lower baseline ARAT scores (median 4 vs. 22 respectively).

TABLE 1
Participant Characteristics

| | Good Recovery (n = 22) | | Poor Recovery (n = 5) | |
|---|---|---|---|---|
| | ΔARAT ≥ 6 | | ΔARAT < 6 | |
| Age, median (IQR) | 62.5 | (52.3-76) | 72 | (55-73) |
| Male, n (%) | 11 | (50%) | 2 | (40%) |
| Female, n (%) | 11 | (50%) | 3 | (60%) |
| Race, n (%) | | | | |
| African American | 18 | (82%) | 5 | (100%) |
| White | 3 | (14%) | 0 | |
| Pacific Islander | 1 | (5%) | 0 | |
| Cardiovascular Comorbidities, n (%) | | | | |
| Atrial Fibrillation | 1 | (5%) | 0 | |
| Congestive Heart Failure | 3 | (14%) | 0 | |
| Hypertension | 19 | (86%) | 4 | (80%) |
| Hyperlipidemia | 14 | (64%) | 2 | (40%) |
| Diabetes | 11 | (50%) | 2 | (40%) |
| Current Smoker | 2 | (9%) | 0 | |
| Stroke Subtype, n (%) | | | | |
| Ischemic Stroke | 20 | (91%) | 5 | (100%) |
| Hemorrhagic Stroke | 2 | (9%) | 0 | |
| Days from stroke to baseline assessment, | 18 | (13.8-19.8) | 20 | (19-22) |
| median (IQR) | | | | |
| Baseline ARAT (0-57), median (IQR) | 22 | (5.3-32.8) | 4 | (3-31) |
| 6 month ARAT (0-57), median (IQR) | 49 | (37.3-57) | 3 | (0-35) |
| ΔARAT, median (IQR) | 20 | (17-31.3) | −3 | (−4-0) |

ARAT = Action Research Arm Test;
IQR = Interquartile range

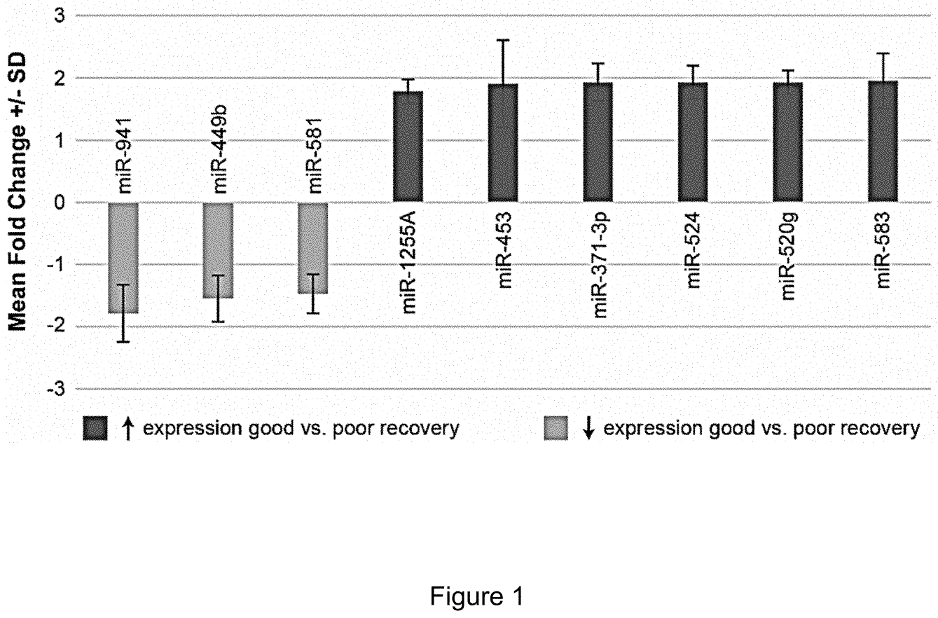

To investigate differences in miRNA expression between the good and poor recovery groups, plasma miRNA expression levels were measured using microarray assays. Nine miRNAs were differentially expressed between the good and poor recovery groups (FIG. 1) out of the 754 miRNAs tested. Six miRNAs showed increased expression -miR-371-3p (p=0.003), miR-524 (p=0.014), miR-520g (p=0.015), miR-1255A (p=0.02), miR-453 (p=0.037), and miR-583 (p=0.046); while three showed decreased expression -miR-941 (p=0.037), miR-449b (p=0.043), and miR-581 (p=0.045). Given the significant imbalance between the good and poor recovery groups, correlational analysis of the significant miRs was also performed, treating ΔARAT as a continuous variable (Table 2). The correlations between ΔARATs for each study participant and miRNA expression levels were in the same direction (positive or negative) as the fold-change for each significant miRNA. MiR-371-3p and miR-941 showed the strongest correlations (0.39 and −0.36 respectively). Pathway analyses revealed that the significant miRNAs primarily converge on pathways associated with cancer, axon guidance, and developmental biology (Table 3).

TABLE 2
Fold-change and false discovery rate (FDR) corrected p-values for miRNA expression
in participants with good (ΔARAT ≥ 6) vs. poor (ΔARAT < 6) recovery of the upper limb.
Correlation between individual ΔARATs and expression levels for each significant miRNA.

| | | | Correlation between |
|---|---|---|---|
| | | FDR-corrected | ΔARAT and miRNA |
| | Fold-change | p-value | expression levels |
| miR-371-3p | 1.93 ↑ | 0.003 | 0.39 |
| miR-524 | 1.93 ↑ | 0.014 | 0.3 |
| miR-520g | 1.93 ↑ | 0.015 | 0.34 |
| miR-1255a | 1.78 ↑ | 0.020 | 0.17 |
| miR-453 | 1.91 ↑ | 0.037 | 0.19 |
| miR-941 | 1.79 ↓ | 0.037 | −0.36 |
| miR-449b | 1.55 ↓ | 0.043 | −0.19 |
| miR-581 | 1.47 ↓ | 0.045 | −0.21 |
| miR-583 | 1.95 ↑ | 0.046 | 0.23 |

ARAT = Action Research Arm Test

TABLE 3
Top ten ranked biological pathways identified for the 9 miRNAs differentially
expressed between participants with good (ΔARAT ≥ 6) vs. poor (ΔARAT < 6) recovery using 3
different miRNA pathway analysis tools.

| | | | Ingenuity Pathway |
|---|---|---|---|
| Rank | miRSystem | mirPath | Analysis |
| 1. | Pathways in Cancer | TGF-beta Signaling | Molecular Mechanisms of |
| | | Pathway | Cancer |
| 2. | Axon Guidance | Signaling Pathways | Axonal Guidance |
| | | Regulating Pluripotency of | Signaling |
| | | Stem Cells | |
| 3. | WNT Signaling Pathway | FoxO Signaling Pathway | G-Protein Coupled |
| | | | Receptor Signaling |
| 4. | Axon Guidance | WNT Signaling Pathway | Protein Kinase A Signaling |
| 5. | Developmental Biology | Oocyte Meiosis | Role of Macrophages, |
| | | | Fibroblasts, and |
| | | | Endothelial Cells in |
| | | | Rheumatoid Arthritis |
| 6. | Role of Calcineurin- | Prostate Cancer | IL-8 Signaling |
| | dependent NFAT Signaling | | |
| | in Lymphocytes | | |
| 7. | Prostate Cancer | Hippo Signaling Pathway | Glucocorticoid Receptor |
| | | | Signaling |
| 8. | ERBB1 Downstream | Central Carbon | Regulation of the |
| | Signaling | Metabolism in Cancer | Epithelial-Mesenchymal |
| | | | Transition Pathway |
| 9. | L1CAM Interactions | Proteoglycans in Cancer | Glioblastoma Multiforme |
| | | | Signaling |
| 10. | MAPK Signaling Pathway | Lysine Degradation | Breast Cancer Signaling by |
| | | | Stathmin1 |

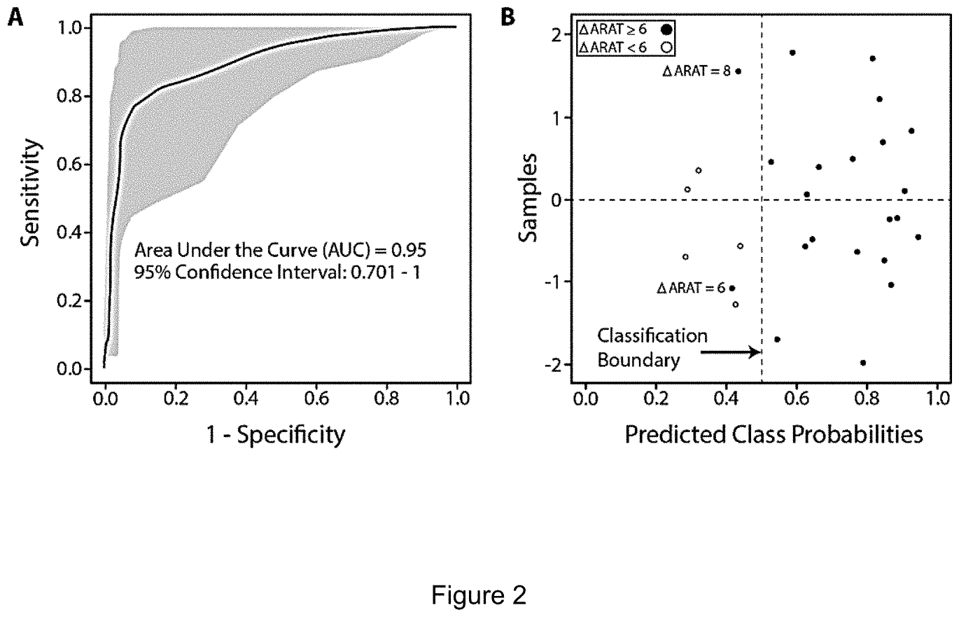

A receiver operating characteristic (ROC) curve analysis was performed to determine whether miRNA biomarkers could accurately predict good versus poor stroke recovery. The five miRNAs with the highest area under the curve (AUC) -miR-581, miR-519b-3p, miR-941, miR-449b, and miR-616 - produced a combined AUC of 0.95 as shown in FIG. 2. Two of these five miRNAs had high AUCs, but were not included in our list of nine differentially expressed miRs in Table 2 due to FDR-corrected p-values>0.05 (miR-519b, p=0.0504; miR-616, p =0.116). The confusion matrix showed that two participants in the good recovery group (ΔARAT≥6) were misclassified into the poor recovery group (ΔARAT<6). The two misclassified participants had the lowest ΔARAT scores among those in the good recovery group.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

What is claimed is:

1. A method of determining if a subject has an increased risk of poor recovery after suffering a stroke, the method comprising
(a) analyzing at least one plasma sample taken from the subject to assess a microRNA (miRNA) profile of the subject
(b) comparing the subject's miRNA profile with a normal miRNA profile, to determine if the subject's miRNA profile is altered compared to a normal miRNA profile,
wherein an alteration of the subject's miRNA profile is indicative that the subject has an increased risk of poor recovery after suffering a stroke.

2. The method of claim 1, wherein the miRNA profile comprises expression levels of at least one of miR-371-3p, miR-524, miR-520g, miR-1255a, miR-453, miR-941, miR-449b, miR-581, miR-583, miR-519b, and miR-616.

3. The method of claim 2, wherein at least one of the miRNAs of miR-371-3p, miR-524, miR-520g, miR-1255a, miR-453, or miR-583 are lower than normal.

4. The method of claim 2, wherein at least one of the miRNAs of miR-941, miR-449b, miR-581, miR-519b, or miR-616 are higher than normal.

5. A method of treating a subject who is recovering from a stroke, the method comprising
(a) determining whether the subject has an increased risk of poor recovery from a stroke by analyzing at least one plasma sample taken from the subject to assess a microRNA (miRNA) profile of the subject and comparing the subject's miRNA profile with a normal miRNA profile, wherein a difference in the subject's miRNA profile compared to a normal miRNA profile is indicative that the subject has an increased risk of poor recovery from the stroke; and
(b) administering a treatment for recovery from the stoke when the subject is determined to have an increased risk of poor recovery from the stroke.

6. A method of treating a subject who is recovering from a stroke, the method comprising
(a) determining whether the subject has an increased risk of poor recovery from a stroke by analyzing at least one plasma sample taken from the subject to assess a microRNA (miRNA) profile of the subject and comparing the subject's miRNA profile with a normal miRNA profile, wherein a difference in the subject's miRNA profile compared to a normal miRNA profile is indicative that the subject has an increased risk of poor recovery from the stroke; and
(b) administering
(i) a treatment effective for poor recovery from the stoke when the subject is determined to have an increased risk of poor recovery from the stroke; or
(ii) a treatment effective for normal or good recovery from the stoke when the subject is determined to not have an increased risk of poor recovery from the stroke.

7. The method of claim 5 or 6, wherein the comparison of the miRNA profile of the subject with the normal miRNA profile may comprise comparing the expression level of miRNAs of the subject's miRNA profile with the expression level of miRNAs of the normal miRNA profile.

8. The method of claim 5 or 6, wherein the miRNA profile comprises expression levels of one or more miRNA selected from miR-371-3p, miR-524, miR-520g, miR-1255a, miR-453, miR-941, miR-449b, miR-581, miR-583, miR-519b, and miR-616.

9. The method of claim 8, wherein the subject is determined to have an increased risk of poor recovery from the stroke when the expression level of at least one of miR-371-3p, miR-524, miR-520g, miR-1255a, miR-453, and miR-583 of the subject's miRNA profile is lower as compared to the expression level of the normal miRNA profile.

10. The method of 8 or 9, wherein the subject is determined to have an increased risk of poor recovery from the stroke when the expression level of at least one of miR-371-3p, miR-524, miR-520g, miR-1255a, miR-453, and miR-583 of the subject's miRNA profile is lower as compared to the expression level of the normal miRNA profile.

11. The method of any one of claims 8-10, wherein the subject is determined to have an increased risk of poor recovery from the stroke when the expression level of at least two of miR-371-3p, miR-524, miR-520g, miR-1255a, miR-453, and miR-583 of the subject's miRNA profile is lower as compared to the expression level of the normal miRNA profile.

12. The method of any one of claims 8-11, wherein the subject is determined to have an increased risk of poor recovery from the stroke when the expression level of at least three of miR-371-3p, miR-524, miR-520g, miR-1255a, miR-453, and miR-583 of the subject's miRNA profile is lower as compared to the expression level of the normal miRNA profile.

13. The method of any one of claims 8-12, wherein the subject is determined to have an increased risk of poor recovery from the stroke when the expression level of at least four of miR-371-3p, miR-524, miR-520g, miR-1255a, miR-453, and miR-583 of the subject's miRNA profile is lower as compared to the expression level of the normal miRNA profile.

14. The method of any one of claims 8-13, wherein the subject is determined to have an increased risk of poor recovery from the stroke when the expression level of at least five of miR-371-3p, miR-524, miR-520g, miR-1255a, miR-453, and miR-583 of the subject's miRNA profile is lower as compared to the expression level of the normal miRNA profile.

15. The method of any one of claims 8-14, wherein the subject is determined to have an increased risk of poor recovery from the stroke when the expression level of each of miR-371-3p, miR-524, miR-520g, miR-1255a, miR-453, and miR-583 of the subject's miRNA profile is lower as compared to the expression level of the normal miRNA profile.

16. The method of any one of claims 8-15, wherein the subject is determined to have an increased risk of poor recovery from the stroke when the expression level of at least one of miR-941, miR-449b, miR-581, miR-519b, or miR-616 of the subject's miRNA profile is higher as compared to the expression level of the normal miRNA profile.

17. The method of any one of claims 8-16, wherein the subject is determined to have an increased risk of poor recovery from the stroke when the expression level of at least two of miR-941, miR-449b, miR-581, miR-519b, or miR-616 of the subject's miRNA profile is higher as compared to the expression level of the normal miRNA profile.

18. The method of any one of claims 8-17, wherein the subject is determined to have an increased risk of poor recovery from the stroke when the expression level of at least three of miR-941, miR-449b, miR-581, miR-519b, or miR-616 of the subject's miRNA profile is higher as compared to the expression level of the normal miRNA profile.

19. The method of any one of claims 8-18, wherein the subject is determined to have an increased risk of poor recovery from the stroke when the expression level of at least four of miR-941, miR-449b, miR-581, miR-519b, or miR-616 of the subject's miRNA profile is higher as compared to the expression level of the normal miRNA profile.

20. The method of any one of claims 8-19, wherein the subject is determined to have an increased risk of poor recovery from the stroke when the expression level of each of miR-941, miR-449b, miR-581, miR-519b, or miR-616 of the subject's miRNA profile is higher as compared to the expression level of the normal miRNA profile. The method of any one of claims 5-20, wherein the normal miRNA profile comprises an miRNA profile of an individual or individuals who experienced a normal or good recovery from a stroke.
